(19) United States
(12) Patent Application Publication
(10) Pub. No.: US 2011/0092432 A1
(43) Pub. Date: Apr. 21, 2011

(54) VASCULAR AGING INHIBITOR AND ANTI-AGING FORMULATION

(75) Inventor: Hikaru Ishii, Tokyo (JP)

(73) Assignee: Hikaru ISHII, Tokyo (JP)

(21) Appl. No.: 12/993,594

(22) PCT Filed: May 19, 2009
(86) PCT No.: PCT/JP2009/059490

§ 371 Date: Nov. 19, 2010

(30) Foreign Application Priority Data
May 20, 2008 (JP) 2008-131621

(51) Int. Cl. A61K 38/39; C07K 14/78; A61P 9/00

(52) U.S. Cl. 514/17.2; 530/300

(57) ABSTRACT

A vascular aging inhibitor includes a low molecular weight collagen derived from fish skin as an essential component, wherein the weight average molecular weight of the low molecular weight collagen derived from fish skin is approximately 3,000.

### TECHNICAL FIELD

The present invention relates to a vascular aging inhibitor and an anti-aging formulation, and relates particularly to a vascular aging inhibitor and an anti-aging formulation that exhibit a high rate of absorption.

### BACKGROUND ART

Metabolic capabilities tend to deteriorate with age, leading to increased fatigue and reduced anatomical functions. Accordingly, in recent years, various tests have been conducted on the transdermal absorption and oral administration of antioxidants (such as vitamins), various hormone preparations, and unsaturated fatty acids as potential anti-aging treatments. However, these tests represent only one step in anti-aging treatment.

On the other hand, collagen represents ⅓ of biological proteins, and is the main structural protein in biostructures. Furthermore, it is now clear that collagen not only performs a simple mechanical function as a support structure for biological bodies, but also has extremely important biological roles in protecting cells and as an intercellular factor.

Accordingly, JP 7-278012 A proposes a metabolic accelerator that comprises a collagen protein or a hydrolysis product thereof as an essential component.

### SUMMARY

Upon aging, as the cells of blood vessel walls age, the blood vessels tend to lose elasticity, and blood platelets and cholesterol accumulate on the blood vessel walls, increasing the possibility of arterial sclerosis.

The present invention provides a vascular aging inhibitor and an anti-aging formulation that suppress blood vessel aging and inhibit arterial sclerosis.

The vascular aging inhibitor and anti-aging formulation of the present invention have the features described below.

(1) According to an aspect of the present invention, there is provided a vascular aging inhibitor comprising a low molecular weight collagen derived from fish skin as an essential component, and further comprising minerals such as calcium and phosphorus, and any of the various vitamins.

As described below, low molecular weight collagen derived from fish skin exhibits a higher rate of absorption than collagen obtained from other raw materials. Accordingly, the favorably absorbed amino acid peptides are reassembled, and the resulting in-vivo collagen promotes metabolism of the blood vessel walls. This causes endothelial cells from the blood vessel walls to peel away, meaning blood platelets and cholesterol adhered to the inner membrane are also gradually removed, thereby reducing the amount of fibrous plaques. As a result, the blood vessel walls are renewed in a substantially constant manner, and the adhesion of blood platelets and cholesterol can be inhibited. By regular administration of a vascular aging inhibitor containing a low molecular weight collagen derived from fish skin as an essential component, the elasticity of the entire blood vessel wall can be improved, and the adhesion of blood platelets and cholesterol can be prevented.

(2) According to another aspect of the present invention there is provided the vascular aging inhibitor disclosed in (1) above, wherein the weight average molecular weight of the low molecular weight collagen derived from fish skin is approximately 3,000.

Compared with a higher molecular weight collagen, the low molecular weight collagen described above is decomposed into amino acid peptides of even lower molecular weight by in-vivo enzymatic decomposition, and therefore the absorption rate within the body, for example via intestinal absorption, is very high, meaning the efficiency of the blood vessel wall regeneration process can be enhanced.

(3) According to yet another aspect of the present invention there is provided an anti-aging formulation in which the vascular aging inhibitor disclosed in (1) or (2) above is in a granular form.

Compared with capsules or tablets, producing the formulation in granular form accelerates the enzymatic decomposition that occurs when the formulation reaches the intestine.

### BRIEF DESCRIPTION OF THE DRAWINGS

Embodiment(s) of the present invention will be described in detail based on the following figures, wherein:

FIG. 1 is a graph describing the relationship between the absorption of fish-derived collagen peptides and animal skin-derived collagen peptides;

FIG. 2 is a diagram illustrating the results of measuring the weight average molecular weight of one example of a collagen derived from fish skin that represents the main component of an anti-aging formulation according to an embodiment of the present invention; and

FIG. 3 is a schematic cross-sectional view of a blood vessel illustrating an example of arterial sclerosis.

### DESCRIPTION OF EMBODIMENTS

One example of a vascular aging inhibitor and an anti-aging formulation of the present invention is described below with reference to the drawings.

Depending on the raw material, collagen can be broadly classified as either animal skin-derived collagen (such as pig skin-derived collagen and chicken skin-derived collagen) and fish-derived collagen. It is well known that animal skin-derived collagen has a higher allergenicity than fish-derived collagen, and therefore fish-derived collagen offers superior safety.

Furthermore, fish-derived collagen includes collagen derived from fish skin and collagen derived from fish scales. Scale-derived collagen is obtained by chemical treatment of the hard fish scales using a strong hydrochloric acid, and therefore not only is there a possibility of the collagen containing residual hydrochloric acid, but because the hydrochloric acid randomly cleaves bioactive amino acid peptide linkages, there is a possibility that a portion of the resulting collagen peptides may have lost its bioactivity. In contrast, fish skin-derived collagen can be decomposed with no loss of the amino acid peptide linkage bioactivity by appropriate application of heat and selection of a specific enzyme, and therefore it is not only safe, but also enables the extraction of collagen peptides that exhibit superior bioactivity to those obtained from scale-derived collagen.

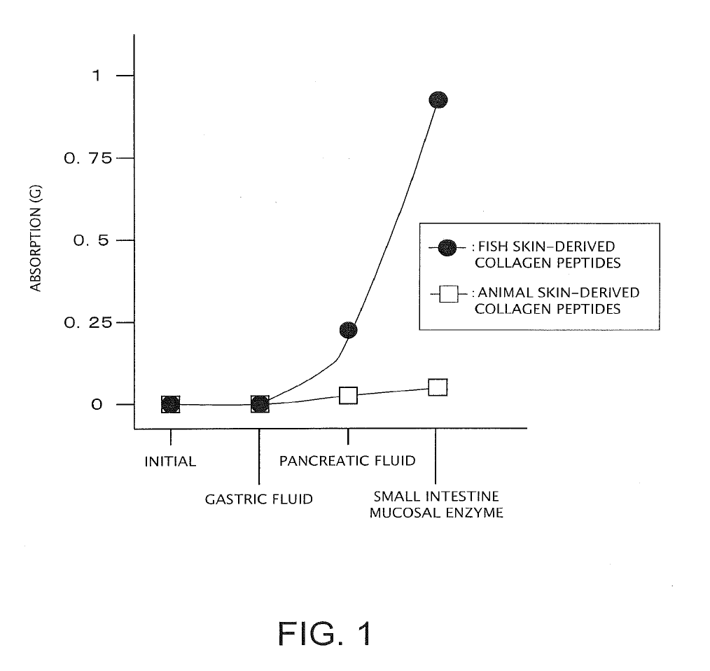

Furthermore, collagen derived from animal skin generally has an amino acid sequence that is peculiar to mammals, and tends to contain a larger amount of the collagen-specific amino acid known as proline than fish-derived collagen. Proline bonds to a hydroxyl group and exists in the form of hydroxyproline within collagen, and has a feature of exhibiting more powerful bonding than other amino acids. Because animal skin-derived collagen contains a much higher amount of hydroxyproline than that found in fish, it tends to be more difficult to decompose. It could be argued that this observation reflects the fact that many mammals are land-based animals, and that the necessity to protect their bodies from the very changeable weather and from foreign bodies resulted in the survival of those mammals for which the skin had been embryologically toughened. Accordingly, as can be seen in FIG. 1, even if an animal-derived collagen and a fish-derived collagen have the same molecular weight, the fish-derived collagen undergoes a much higher degree of decomposition than the animal-derived collagen. In other words, fish-derived collagen exhibits an extremely high in-vivo absorption rate (see FOOD Style 21, 2003.2, pp. 85 to 88).

However, in-vivo collagen is usually generated in three stages known as premature cross-linking, mature cross-linking and aged cross-linking. In this description, “premature cross-linking” refers to a state of intramolecular and intermolecular collagen cross-linking which exists in a high proportion in the collagen fibers of fetuses or newly born animals and which is readily dissolved by acid, “mature cross-linking” refers to cross-linking which is stable in the presence of acid or heat, is not reduced by sodium borohydride, and includes cross-linked structures such as pyridinoline or deoxypyridinoline, and “aged cross-linking” refers to a state in which cross-linking has progressed further via the Maillard reaction (also known as a glycation reaction) and under the influence of in-vivo active enzymes (see “The Collagen Story”, by Daisaburo FUJIMOTO, published by Tokyo Kagaku Dozin Co., Ltd., 3rd edition, Jul. 3, 2006, pp. 73 to 100).

Generally, as aging proceeds, the proportion of aged cross-linking within the collagen that constitutes blood vessel walls tends to increase. As a result, the degree of collagen cross-linking in the blood vessel walls increases, which causes the blood vessel walls to harden, causes a loss in elasticity of the blood vessels themselves, and increases the accumulation of blood platelets and cholesterol on the blood vessel walls, making arterial sclerosis more likely.

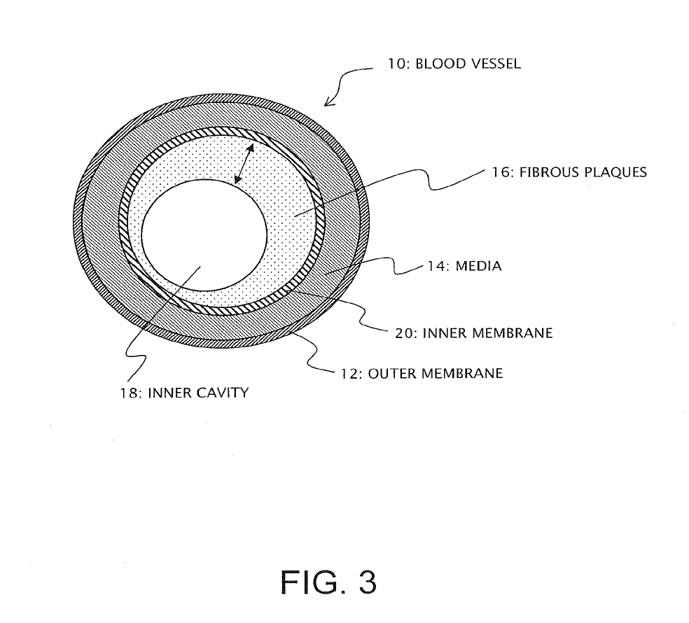

For example, FIG. 3 schematically illustrates a state in which arterial sclerosis has caused thickening and hardening of an artery wall. As can be seen in FIG. 3, in an artery wall suffering from atherosclerosis, the smooth muscle cells in the vicinity of an inner membrane 20 that exists on the inside of an outer membrane 12 and media 14 of a blood vessel 10 undergo repeated division, and the resulting monocytes permeate and are accumulated within the endothelial cells, and both the monocytes and the smooth muscle cells then start phagocytosis of fats contained within the blood, mainly cholesterol, causing the endothelial cells to also start accumulating lipids. As a result, the endothelial cells are enlarged, and when the endothelial cell layer cracks, blood platelets begin to bond to the exposed collagen fibers, resulting in the generation and accumulation of fibrous plaques 16 which are fatty structures that protrude into an inner cavity 18 of the blood vessel 10. The thickness of these accumulated fibrous plaques 16 is indicated by an arrow in FIG. 3. Generally, atherosclerosis is treated by using a vascular catheter to perform a balloon angioplasty and position a stent within the blood vessel. However, preventing occurrence of the above type of atherosclerosis is desirable. Current medications act by reducing vascular resistance to achieve an antihypertensive effect, or by reducing blood lipids to ameliorate hyperlipidemia, but no current medications can be claimed to act directly upon the blood vessel to provide a mechanism that achieves restoration and regeneration of the blood vessel.

Accordingly, the vascular aging inhibitor of the present embodiment includes collagen as an essential component.

As described above, low molecular weight collagen derived from fish skin exhibits a higher rate of absorption than collagen obtained from other raw materials, and therefore the favorably absorbed amino acid peptides are reassembled, and the resulting in-vivo collagen promotes metabolism of the blood vessel walls. This accelerates the metabolism of the endothelial cells on the inner cavity 18 side of the media 14 of the type of blood vessel 10 shown in FIG. 3, and the resulting turnover of the cells on the inner cavity 18 side of the inner membrane causes these cells to peel away, meaning the fibrous plaques 16 formed on the inner membrane are also removed and flushed into the bloodstream, where they can be treated as waste products and expelled from the body.

Moreover, by orally administering the vascular aging inhibitor of the present embodiment over a period of time, metabolism of the cells of the blood vessel wall can be accelerated, resulting in an improvement in the elasticity of the entire blood vessel wall and a dramatic suppression of the generation of fibrous plaques 16.

In addition, in the vascular aging inhibitor according to the present embodiment, the weight average molecular weight of the aforementioned low molecular weight collagen derived from fish skin is approximately 3,000. Accordingly, compared with higher molecular weight collagen, the collagen is decomposed into amino acid peptides of even lower molecular weight by in-vivo enzymatic decomposition, and therefore the absorption rate within the body, for example via intestinal absorption, is very high, meaning the efficiency of the blood vessel wall regeneration process can be enhanced.

Examples of fish from which the fish-derived collagen can be obtained include bonito, tuna, marlin, cod, horse mackerel, mackerel, salmon, trout, saury, eel, tilapia, thread-sail filefish, grouper, halibut, flounder, sole, herring, sardine, sharks, rays, blowfish, yellow tail, scorpion fish, rockfish and pollock, and use of fish skin collagen from one of these fish is preferred.

Examples of the protein hydrolyzing enzyme that can be used for the collagen decomposition include neutral protease, alkaline protease, acidic protease and pepsin. The enzymatic decomposition is typically conducted by incubating the collagen in an acidic environment (for example a pH of 1.0 to 2.0) at a temperature within a range from 30° C. to 60° C., and preferably from 40° C. to 50° C., for a period of 10 to 60 minutes, and preferably 15 to 40 minutes.

The enzymatic decomposition product obtained as a result of the above enzymatic decomposition is subjected to a reverse osmosis membrane treatment and recovered as a concentrated liquid. For the reverse osmosis membrane, a membrane having a salt blocking ratio of 10% to 50% is preferred, and examples of commercially available membranes include the products NTR-7410, NTR-7430 and NTR-7450 (all manufactured by Nitto Denko Corporation). During concentrating of the liquid, water is preferably added in an amount equivalent to 1 to 10 times the original liquid volume while the liquid is passed through the membrane.

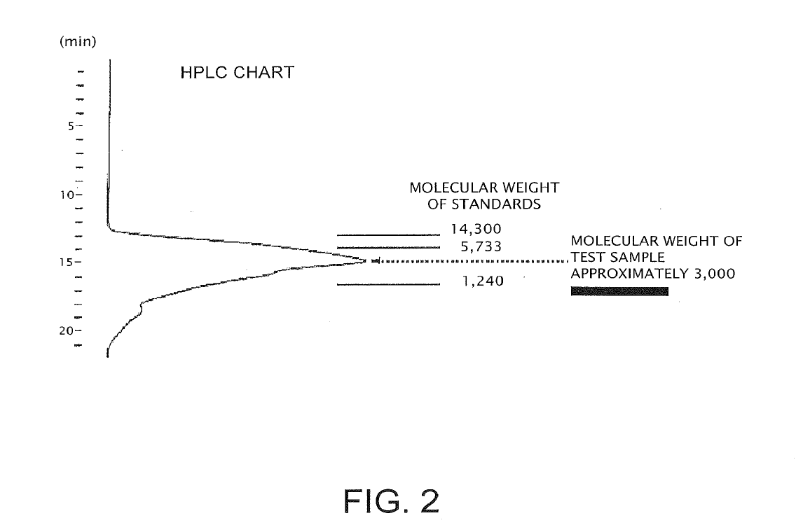

FIG. 2 shows the result of measuring the weight average molecular weight of the fish skin-derived low molecular weight collagen contained within the vascular aging inhibitor of the present embodiment. The measurement conditions involved using an HPLC analysis method under the conditions described below.

Column: TSK-gel guard column PWXL, TSK-gel G3000 PWXL, TSK-gel G2500 PWXL (all manufactured by Tosoh Corporation)

Mobile phase: 0.5% (w/v) NaCl aqueous solution

Flow rate: 0.8 ml/min.

Temperature: room temperature (23° C.)

Detector: ultraviolet spectrometric detector (220 nm)

Sample: injection of 10 μl of a 1 to 2% (w/v) aqueous solution of fish skin-derived collagen

Standard sample: injection of 10 μl of an aqueous solution containing 1% (w/v) of each of three standards, namely lysozyme (derived from chicken egg white, weight average molecular weight: 14,300), insulin (derived from bovine pancreas, weight average molecular weight: 5,733), and bradykinin, weight average molecular weight: 1,240) (all manufactured by Sigma-Aldrich Co.).

Furthermore, an anti-aging formulation according to the present embodiment is a formulation that contains the vascular aging inhibitor described above in a granular form. Compared with capsules or tablets, producing the formulation in granular form accelerates the enzymatic decomposition that occurs when the formulation reaches the intestine.

### EXAMPLES

A description of the vascular aging inhibitor and the anti-aging formulation of the present invention using a working example is presented below. It should be noted that the scope of the present invention is in no way limited by the example described below.

### Preparation of Granular Anti-Aging Formulation

Using the skin from gadiformes or pleuronectiformes as the raw material for the fish skin-derived collagen, and using pepsin as the decomposition enzyme, the pH was adjusted to 1.5, and an enzymatic decomposition was then conducted for 20 minutes at a temperature of 40° C. Subsequent measurement of the product under the conditions described above for the HPLC analysis method revealed a weight average molecular weight for the obtained fish skin-derived collagen of 3,000.

### Test Example

The granular anti-aging formulation obtained from the above preparation was administered orally once per day to 12 test subjects, in an amount sufficient to provide 5.0 g of the fish skin-derived collagen per administration. The state of the blood vessels in each of the 12 test subjects (labeled “test subject 1” to “test subject 12” in Table 1) was analyzed prior to commencing administration, and then after 3 months of administration, by using an ultrasound apparatus (LOGIQ 7 PRO, manufactured by GE Co.) to measure the thickness of the inner membrane of the carotid artery.

TABLE 1

| | | | Prior to administration | | | | After 3 months of administration | | | |
|---|---|---|---|---|---|---|---|---|---|---|
| | | | Carotid artery | Carotid artery | | | Carotid artery | Carotid artery | | |
| | | | inner right side | inner left side | | | inner right side | inner left side | | |
| | Age | | membrane | membrane | TC | LDL-C | membrane | membrane | TC | LDL-C |
| | (years) | Gender | thickness (mm) | thickness (mm) | (mg/dl) | (mg/dl) | thickness (mm) | thickness (mm) | (mg/dl) | (mg/dl) |
| Test | 71 | F | 0.75 | 0.87 | 280 | 241 | 0.5 | 0.7 | 263 | 172 |
| subject 1 | | | | | | | | | | |
| Test | 50 | M | 0.66 | 0.75 | 228 | 158 | 0.51 | 0.62 | 235 | 145 |
| subject 2 | | | | | | | | | | |
| Test | 67 | M | 0.89 | 1.04 | 277 | 240 | 0.85 | 0.76 | 209 | 118 |
| subject 3 | | | | | | | | | | |
| Test | 50 | M | 0.92 | 1 | 258 | 170 | 1.03 | 1.03 | 302 | 218 |
| subject 4 | | | | | | | | | | |
| Test | 58 | M | 0.7 | 0.7 | 148 | 53 | 0.73 | 0.65 | 157 | 67 |
| subject 5 | | | | | | | | | | |
| Test | 61 | M | 0.7 | 1.45 | 317 | 227 | 0.62 | 1.35 | 282 | 199 |
| subject 6 | | | | | | | | | | |
| Test | 66 | F | 0.8 | 1.3 | 252 | 138 | 0.75 | 1.03 | 277 | 167 |
| subject 7 | | | | | | | | | | |
| Test | 48 | M | 0.73 | 0.95 | 272 | 177 | 0.62 | 0.75 | 270 | 170 |
| subject 8 | | | | | | | | | | |
| Test | 56 | M | 0.7 | 1.55 | 297 | 218 | 0.62 | 1.25 | 283 | 208 |
| Subject 9 | | | | | | | | | | |
| Test | 71 | M | 1.3 | 1.59 | 205 | 130 | 1.17 | 1.44 | 197 | 127 |
| subject 10 | | | | | | | | | | |
| Test | 59 | M | 0.7 | 1.1 | 191 | 118 | 0.7 | 1.03 | 195 | 127 |
| subject 11 | | | | | | | | | | |
| Test | 64 | M | 1.37 | 1.24 | 245 | 155 | 1.24 | 0.82 | 248 | 167 |
| subject 12 | | | | | | | | | | |

Notes)
TC: total cholesterol, LDL-C: low molecular weight cholesterol (commonly called “bad cholesterol”), inner membrane thickness values are measured values from the right and left sides of an ultrasound echo image.

Arterial sclerosis is a phenomenon in which the inner membrane of the artery thickens, and if the inner membrane thickness exceeds 1 mm, then arterial sclerosis is diagnosed regardless of age. Currently, it is considered that the most accurate method for diagnosing arterial sclerosis involves measurement of the thickness of the inner membrane of the carotid artery using a precise ultrasound apparatus.

The 12 test subjects included 10 men and 2 women, with ages from 48 to 71 years, and an average age of 60.1 years. Those patients who were taking medication for hyperlipidemia stopped taking their medication one month prior to beginning the test, and were administered only the formulation of the present invention during the test period. As is evident from Table 1, as a result of the oral administration of the anti-aging formulation prepared in the example over a 3 month period, 10 of the 12 test subjects exhibited a clear reduction in the thickness of the carotid artery inner membrane, and the thickness of fibrous plaque also decreased.

Furthermore, a reduction in total cholesterol was confirmed in 4 of the 12 test subjects, and a reduction in LDL cholesterol was confirmed for 6 of the test subjects.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The exemplary embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

1. A vascular aging inhibitor comprising a low molecular weight collagen derived from fish skin as an essential component, wherein a weight average molecular weight of the low molecular weight collagen derived from fish skin is approximately 3,000.

2. The vascular aging inhibitor according to claim 1, wherein
the low molecular weight collagen derived from fish skin is obtained by enzymatic decomposition using skin of gadiformes or pleuronectiformes as a raw material.

3-4. (canceled)

5. An anti-aging formulation, comprising the vascular aging inhibitor according to claim 1 in a granular form.

6. An anti-aging formulation, comprising the vascular aging inhibitor according to claim 2 in a granular form.

7-8. (canceled)
